(19) United States
(12) Patent Application Publication
(10) Pub. No.: US 2025/0281317 A1
(43) Pub. Date: Sep. 11, 2025

(54) Flexible Cock Ring for Male Genitalia

(71) Applicant: Erik Matthew Louderback, Woodstock, GA (US)

(72) Inventor: Erik Matthew Louderback, Woodstock, GA (US)

(21) Appl. No.: 18/598,016

(22) Filed: Mar. 7, 2024

(51) Int. Cl. A61F 5/41

(52) U.S. Cl. CPC A61F 5/41; A61F 2005/414

(57) ABSTRACT

An innovative Flexible Cock Ring for Male Genitalia that grants user sole authority to repurpose the common accessory—the wristband—used in various applications to encircle around penis or penis/scrotum combination and developed from any material, ensuring a personalized experience. The device's extensive applications, including exclusive rights for medical use, position it as a comprehensive and unmatched solution in the field. It provides flexibility, comfort, sexual stimulation, bulge enhancement, male genitalia support and fashionable appeal to the user. Furthermore, its unparalleled comfort and relief for individuals recovering from testicular surgery is an exclusive claim, highlighting its therapeutic role. Additionally, it includes exclusive rights to the use of flexible plastic-derived synthetic, non-synthetic, rubber or elastic inner material to establish it as a comfortable choice. The outer fabric composition claim secures exclusive rights to the use of nylon and synthetic and non-synthetic fibers, ensuring unmatched adaptability.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

This invention relates to devices commonly known as cock rings or penis bands, specifically focusing on the Flexible Cock Ring for Male Genitalia designed to safeguard the utilization of wristbands or similar flexible bands crafted from suitable materials including but not limited to rubber, plastic-derived, silicone or elastic inner material. These bands are intended for enhancing or providing support to male genitalia, encompassing various applications, such as sexual stimulation and medical purposes, among others.

### Description of the Related Art

Cock rings, also known as penile rings or love rings, are devices designed to encircle the base of the penis or penis and scrotum, typically restricting blood flow to create a firmer and longer-lasting erection. Their importance lies in their ability to enhance sexual performance and pleasure for both partners. By maintaining blood in the penis, cock rings can help achieve and sustain erections, potentially alleviating issues such as erectile dysfunction. Additionally, they can intensify sensations and contribute to a more satisfying sexual experience.

Facts and figures regarding the use of cock rings underscore their widespread popularity and acceptance. Many individuals and couples incorporate these devices into their intimate activities. According to various surveys, a significant percentage of men and women report positive experiences and increased satisfaction when using cock rings. The market for these products has seen growth, with a diverse range of designs and materials catering to different preferences and needs.

Advancements in the field of cock rings have focused on enhancing comfort, safety, and versatility. Modern materials such as medical-grade silicone and adjustable designs contribute to a more user-friendly experience. Some innovative designs incorporate additional features, such as vibrating elements or textured surfaces, to further enhance pleasure. As technology continues to evolve, there is ongoing research and development aimed at creating smart cock rings that may integrate with digital devices or provide real-time feedback for both partners.

Despite advancements, there is a need for further research and development in the field of cock rings. This includes exploring more personalized and customizable options to cater to diverse anatomies and preferences. Safety and health considerations are paramount, prompting ongoing efforts to refine materials and designs to minimize risks. Public awareness and education regarding the proper use of cock rings, potential benefits, and safety precautions play a crucial role in promoting responsible and enjoyable use within intimate relationships.

Through researching prior art, multiple advancements have been seen in the similar field. Several patents exist in the field related to devices for male genitalia enhancement or support, particularly in the category of cock rings. One example is the patent titled “Adjustable Constriction Ring for Improving Erectile Dysfunction” (U.S. Pat. No. 9,123,060). This patent describes a cock ring with an adjustable mechanism to control constriction, allowing users to customize the level of pressure based on their comfort and needs. The invention aims to address issues related to erectile dysfunction by providing a more adaptable and user-friendly solution.

Another noteworthy patent is “Vibrating Penile Ring” (U.S. Pat. No. 8,142,379), which focuses on incorporating vibrating elements into a penile ring. This innovation aims to enhance pleasure for both partners during sexual activity. The patent details the design and functionality of a vibrating penile ring that can be easily worn and operated, contributing to a more stimulating experience.

In the realm of advancements, the patent titled “Smart Cock Ring with Biometric Feedback” (U.S. Pat. No. 10,654,789) represents a more recent development. This patent describes a cock ring that goes around the penis, equipped with sensors to monitor various biometric parameters, providing real-time feedback to users. The smart features may include data on heart rate, body temperature, or other physiological indicators, adding a new dimension to the user experience and potentially contributing to sexual health monitoring.

While these patents showcase innovation in the field, ongoing research and development are necessary. Patents related to personalized and customizable cock ring designs, advanced materials with improved safety profiles, and more seamless integration of technology into these devices are areas where future advancements may occur. As technology and understanding of human physiology progress, further patents are likely to emerge, shaping the landscape of male genitalia enhancement and support devices.

There are multiple solutions that have been presented in prior art. However, these solutions are limited and restricted to their conventional architecture, styling, installation system and have considerable shortcomings, which adversely affect the convenience and appeal with which they can be used. The prior systems have certain limitations including the design of assemblies such as restriction, which make them not suitable for every type of user. Moreover, the ease of use of these assemblies is indeterminate.

The current invention introduces an innovative Flexible Cock Ring for Male Genitalia that encircles the penis or penis/scrotum combination in a flexible and versatile format marking a significant advancement in the field. This product brings attention to the repurposing of commonly available items like wristbands or similar flexible bands into a functional accessory for enhancing male genitalia. By utilizing materials such as rubber, plastic-derived compounds, silicone, or elastic inner material, the versatility of this cock ring is underscored, allowing for customization according to size and preference. Its primary aim is to ensure the safe and effective utilization of these materials for supporting and enhancing male genitalia function, spanning various applications from sexual enhancement to medical use.

This invention addresses a critical need for a versatile solution that caters to a range of purposes concerning male genitalia. Beyond its role in sexual enhancement, the Flexible Cock Ring for Male Genitalia extends its utility to provide support and aid in medical contexts. The adaptability of materials and sizes ensures a broad applicability, allowing individuals to find the most suitable option for their specific needs. With a focus on safety and efficacy, this patent opens avenues for innovation in the realm of male genitalia accessories, promising enhanced functionality, comfort and appeal for users across different scenarios.

None of the previous inventions and patents, taken either singularly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary desirable objective of the invention is to provide an innovative Flexible Cock Ring for Male Genitalia that encircles around penis or penis/scrotum combination offering versatility by repurposing a common item—the wristband and developed from variety of suitable materials—into a multifunctional device for male genitalia to provide support and enhancement.

It is an objective of the invention to provide a customizable assembly that makes it flexible, not rigid or restricting like most cock rings, thus suitable for a wide range of users, accommodating different anatomies and preferences.

It is an objective of the invention to provide enhanced comfort by utilizing a variety of outer materials including, but not limited to rubber, nylon, fabric, soft materials or elastic inner material, the invention focuses on enhancing comfort during use, addressing a key consideration for individuals seeking genitalia support or enhancement devices.

It is an objective of the invention to allow for the use of a variety of outer materials including, but not limited to nylon, rubber, soft materials or elastic materials, providing users with options to choose the most comfortable and suitable material and designs for their preferences.

It is an objective of the invention to provide custom sizing: wherein with multiple lengths, widths, circumferences, and diameters available, users can select a cock ring that fits securely, ensuring both comfort and effectiveness in enhancing or supporting the penis and scrotum.

It is an objective of the invention to ensure that the invention's protective scope extends to medical use, by providing genital support after testicular surgeries to provide comfort, unlike restricting apparatuses, including potential benefits for individuals with specific medical conditions related to the male genitalia.

It is an objective of the invention to help lift genitalia to create the perception of a larger enhanced bulge. The tension of the flexible band in the proposed variation of encircling both penis and scrotum can push the male genitalia forward, thus increasing the perception of a larger enhanced bulge.

Other aspects, advantages and novel features of the present invention will become apparent from the detailed description of the invention when considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The accompanying figures, whereas reference numerals refer to identical or functionally similar elements throughout the separate views, together with the detailed description below, are incorporated herein and form part of the specification, and serve to further illustrate embodiments of concepts that include the claimed invention and explain various principles and advantages of those embodiments.

FIG. 1 shows the front view of the Flexible Cock Ring for Male Genitalia as the preferred embodiment of the invention.

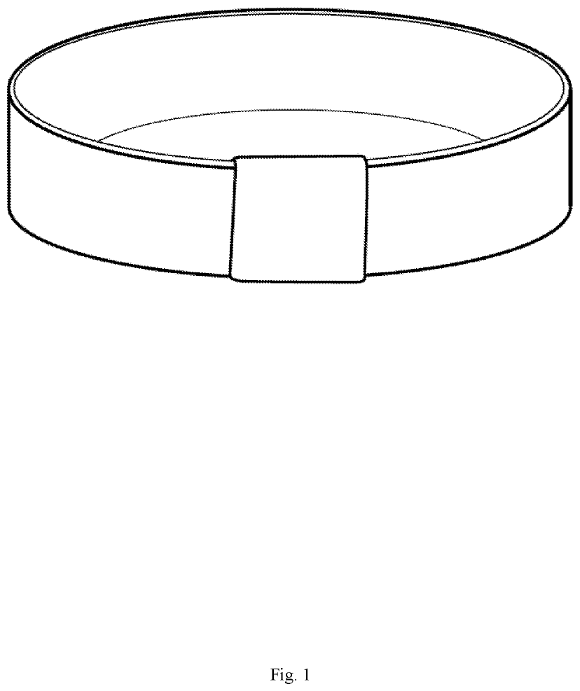

FIG. 2 shows back view of the Flexible Cock Ring for Male Genitalia as the preferred embodiment of the invention.

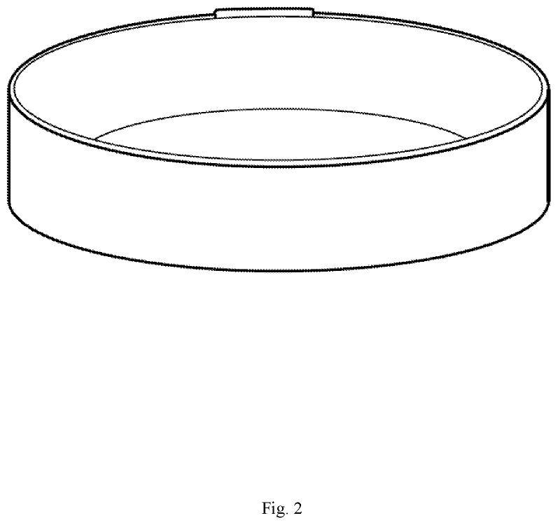

FIG. 3 shows the perspective view of the Flexible Cock Ring for Male Genitalia encircling both the penis and scrotum combination as the preferred embodiment of the invention.

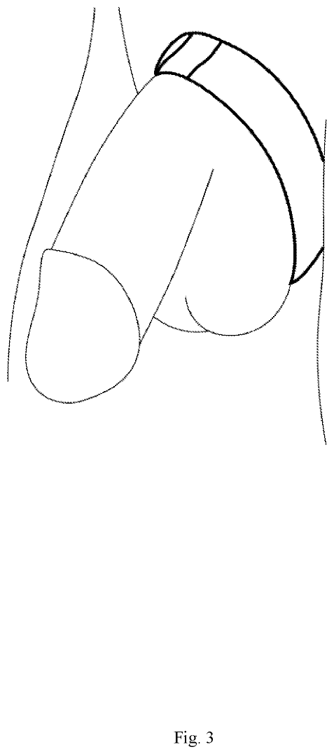

FIG. 4 shows the perspective view of the Flexible Cock Ring for Male Genitalia encircling the penis only as the secondary embodiment of the invention.

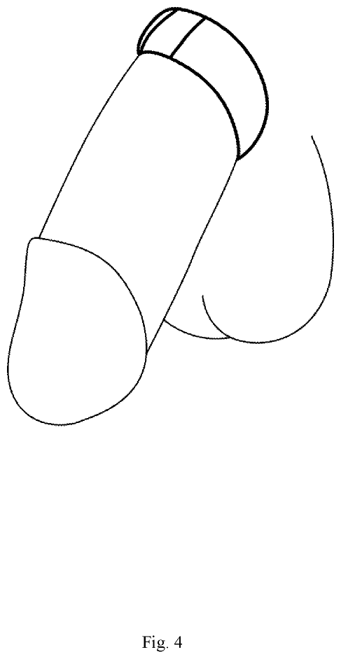

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention, so as not to obscure the disclosure with details that will be readily apparent to those of ordinary skill in the art having the benefit of the description herein.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present invention is directed to a Flexible Cock Ring for Male Genitalia, which is a revolutionary approach to male genitalia enhancement and support by ingeniously repurposing a common item—the wristband. The patent details specifically underscore the flexibility embedded in this invention, as compared to rigid or restrictive cock rings, allowing for the use of any material and any size. This adaptability ensures a customizable experience, catering to individual preferences and needs, marking a notable departure from conventional, one-size-fits-all solutions.

At the heart of this inventive concept is the overarching intent to safeguard the use of wristbands or similar flexible bands encircling the penis or penis/scrotum combination made from rubber, silicone, plastic-derived materials or elastic inner material for male genitalia enhancement or support. This encompasses a broad spectrum of applications, emphasizing a commitment to not only sexual health, sexual enhancements and genital support, but also potential medical uses. By embracing versatility, the Flexible Cock Ring for Male Genitalia emerges as a comprehensive solution, positioning itself as a groundbreaking device in the field.

Beyond its primary focus on sexual enhancement, the patent recognizes the diverse applications of the Flexible Cock Ring for Male Genitalia. It highlights instances where customers seek comfort or relief, particularly those recovering from testicular surgery. The use of rubber, plastic-derived, silicone or elastic inner material becomes significant in this context, emphasizing the potential therapeutic role in the flexible composition of the device in post-surgical scenarios. This underscores the compassion and thoughtfulness embedded in the invention's design, acknowledging the broader spectrum of user needs.

Delving into the specifics of the outer material composition, the patent notes that the current fabric is nylon, but envisions the possibility of using other synthetic and non-synthetic fibers. This forward-thinking approach demonstrates an awareness of evolving technological capabilities and user preferences. The willingness to explore alternative materials ensures the Flexible Cock Ring for Male Genitalia remains adaptable to advancements, potentially offering enhanced durability, comfort, or other desirable attributes.

Furthermore, the patent recognizes the dynamic nature of user preferences by acknowledging that the sizing of band dimensions, such as length, width, circumference and diameter, may differ over time. This foresight reflects a commitment to continuous improvement and user satisfaction, emphasizing the inventor's dedication to creating a device that not only meets current needs, but evolves with changing expectations and preferences in the realm of male genitalia enhancement and support.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. A Flexible Cock Ring for Male Genitalia comprising a repurposed wristband or similar flexible band, uniquely adapted to provide enhancement, comfort, and support to male genitalia.

2. The Flexible Cock Ring for Male Genitalia of claim 1, providing a customizable assembly that is flexible in its composition, thus suitable for a wide range of users, accommodating different anatomies and preferences.

3. A Flexible Cock Ring for Male Genitalia of claim 1, constructed from inner materials such as rubber, plastic-derived compounds, silicone, or elastic inner material, ensuring optimal comfort and support during use, particularly beneficial for post-surgical recovery.

4. A Flexible Cock Ring for Male Genitalia of claim 1, incorporates an outer fabric composition comprising, but not limited to, nylon, with the intention to explore the use of other synthetic and non-synthetic fibers, thereby ensuring adaptability and innovation in design.

5. A Flexible Cock Ring for Male Genitalia of claim 1, wherein the size variability in terms of length, width, circumference, or diameter allows for a tailored fit to accommodate a wide range of individual anatomies, ensuring maximum effectiveness and comfort for users.

6. The Flexible Cock Ring for Male Genitalia of claim 1, featuring a design optimized for post-testicular surgery recovery, offering exceptional comfort, flexibility, and therapeutic benefits for individuals with specific medical conditions related to the male genitalia.

7. The Flexible Cock Ring for Male Genitalia of claim 1, offering a versatile solution for the purpose of enhancing male genitalia by increasing the perceived bulge size, while providing support and comfort in various contexts, including but not limited to sexual activity, medical recovery, and everyday wear.

8. The Flexible Cock Ring Male Genitalia of claim 1, featuring a material composition that promotes airflow and circulation to the male genital area, enhancing comfort and reducing the risk of discomfort or irritation during prolonged wear.

9. A Flexible Cock Ring for Male Genitalia of claim 1, featuring a material composition that is hypoallergenic and skin-friendly, minimizing the risk of allergic reactions or irritation, thus ensuring safety and comfort for users with sensitive skin.

10. The Flexible Cock Ring for Male Genitalia of claim 1, designed with ease of use and maintenance in mind, allowing for effortless application, adjustment, and cleaning for convenient and hygienic use.

11. A Flexible Cock Ring for Male Genitalia of claim 1, featuring a durable construction that withstands repeated use, ensuring longevity and reliability for extended periods of wear.

12. The Flexible Cock Ring for Male Genitalia of claim 1, offering a discreet and aesthetically pleasing design, allowing for comfortable and confident wear in various settings.
